(19) United States
(12) Patent Application Publication
(10) Pub. No.: US 2015/0284669 A1
(43) Pub. Date: Oct. 8, 2015

(54) METHODS OF CULTURING CELLS OR TISSUES AND DEVICES FOR CELL OR TISSUE CULTURE

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Deepak Choudhury, Singapore (SG); Chukwuemeka Anene-Nzelu, Singapore (SG); Hanry Yu, Singapore (SG); Yi-Chin Toh, Singapore (SG); Hwa Liang Leo, Singapore (SG); Sum Huan Ng, Singapore (SG)

(21) Appl. No.: 14/441,831

(22) PCT Filed: Nov. 8, 2012
(86) PCT No.: PCT/SG2012/000424

§ 371 Date: May 8, 2015

(51) Int. Cl. C12M 1/12

(52) U.S. Cl. CPC C12M 25/06

(57) ABSTRACT

A method of culturing cells or tissues is provided. The method comprises a) providing a support, wherein the support is an optical medium with a patterned surface; b) contacting the support with cells and a culture medium; and c) culturing the cells under suitable cultivation conditions. A device for cell or tissue culture comprising an optical medium, wherein a patterned surface of the optical medium forms a support upon which cells are cultivated, is also provided.

### TECHNICAL FIELD

The invention relates to methods of culturing cells or tissues, and devices for cell or tissue culture.

### BACKGROUND

Surface modified substrates have been explored for purposes of orienting and aligning cells in a particular direction for tissue engineering and mechanobiology studies. Methods for modifying a surface of a substrate may be broadly classified as chemical surface modification and physical surface modification.

In chemical surface modification, surface of substrates such as polystyrene (PS) may be modified by a chemical reagent using processes such as negative-silver-ion implantation, oxygen-plasma treated stripes, and phospholipid bilayers, with object of orienting cells grown on the substrates. However, chemical surface modification methods require rigorous processing conditions that are usually complicated or tedious to perform. Furthermore, processes such as chemical oxidation or corona may result in formation of a milieu of functional groups on the surface (instead of just one intended functional group), which leads to non-specific reactions of the cells on the substrate surface.

Physical surface modification, on the other hand, includes patterning a surface of a substrate to modify its surface topography for cell culture. Methods to form patterns on a substrate surface include ultraviolet-laser irradiation, UV embossing, nanoimprinting, electron-beam lithography, photolithography, and proton beam micromachining. All of these methods involve costly clean-room equipment and extensive protocols, which are not easily available to small scale companies and research labs for cell-based screening or research applications. Furthermore, highly skilled labor is required to carry out the methods, in particular, for creation of large area grooved substrates (especially nano grooves) which presents a significant technological challenge.

In view of the above, there is a need for an improved method of providing a patterned surface for culturing cells or tissues that addresses at least one of the above mentioned problems.

### SUMMARY OF THE INVENTION

In a first aspect, the invention refers to a method of culturing cells or tissues. The method comprises
a) providing a support, wherein the support is an optical medium with a patterned surface;
b) contacting the support with cells and a culture medium; and
c) culturing the cells under suitable cultivation conditions.

In a second aspect, the invention refers to a device for cell or tissue culture. The device comprises an optical medium, wherein a patterned surface of the optical medium forms a support upon which cells are cultivated.

### BRIEF DESCRIPTION OF THE DRAWINGS

The invention will be better understood with reference to the detailed description when considered in conjunction with the non-limiting examples and the accompanying drawings, in which:

FIG. 1 is a schematic diagram showing a method to fabricate a device for cell or tissue culture (termed herein as “gratings on a dish”) according to various embodiments. A support, which may be in the form of an optical medium having a patterned surface, such as CD-R, DVD-R and holographic gratings, may be cut or diced into smaller pieces according to the dimensions of the cell culture wells and dishes. For example, the culture dishes may have a diameter ranging from 35 mm to 150 mm, such as 35 mm, 60 mm, 100 mm and 150 mm. Accordingly, the support may be sized to fit into the respective culture dish by, for example, having a diameter that is equal to or smaller than the diameter of the culture dish. Even though cell culture wells and dishes are generally round in shape, the support may assume other shapes which allow it to be accommodated by the culture well/dish. In various embodiments, the optical medium comprises a grating such as a diffraction grating. One or more supports may be subjected to further processing, such as washing with organic solvents and/or an aqueous solution, and placed inside the wells/dishes to complete the device.

FIG. 2 is a series of photographs depicting processing of a surface of CD-R to form gratings on a dish according to an embodiment. In (A), an aluminium foil layer is scratched at a corner (not discernible in the photograph) and an adhesive tape is pressed against the scratched region of the aluminium layer. In (B), the aluminium layer on the CD-R surface is peeled off to reveal or to expose the underlying polycarbonate layer. In (C), the CD-R is cut into smaller pieces. For example, the size of the pieces may have dimensions of 1 cm×1 cm for 24-well plates, or 1.5 cm×1.5 cm for 12-well plates. (D) shows a cut-out piece being washed with a solvent such as methanol to clean the surface of dirt, chemical and/or dyes. In (E), the cut-out pieces are dried. In (F), the dried cut-out pieces are washed with a disinfectant such as ethanol. In (G), the cut-out pieces are autoclaved (105° C., 21 mins) to render the surface of the pieces sterile. In (H), the sterile CD-R cut-out pieces are placed in well plates to complete the “gratings on a dish” device. In (I), cells are seeded on top of the gratings along with a culture medium.

FIG. 3 are (A) and (B) Scanning Electron Microscopy (SEM) images; and (C) and (D) Atomic Force Microscopy (AFM) images showing topography of the diffraction gratings on CD-R, DVD-R, and holographic gratings. In (A) and (B), SEM images of the CD-R and DVD-R show grooves and ridges on the surface. For CD-R, both the aluminium foil layer (top image) and the polycarbonate layer (bottom image) contained grooves. For DVD-R, the inside surface of the polycarbonate had clear groove patterns. In (C) and (D), AFM images of the holographic gratings show grooves and ridges on the surface. The stripe width and periodicity of the gratings were measured and found to be similar to that provided by the manufacturer (Edmund Optics). Stripe depth measured about 300 μm (500 lines/mm) and about 200 μm (1000 lines/mm) for the two different gratings.

FIG. 4 depicts examples of diffraction gratings of different groove shapes. (Source: http://www.horiba.com/fileadmin/uploads/Scientific/Documents/Gratings/New_gratings_catalogue_{—}2011.pdf) (A1) Sinusoidal formed by holographic recording; (A2) initial pseudo sinusoidal formed by holographic recording; (A3) triangular holographically recorded and ion etched groove profile; (A4) diamond ruled sawtooth profile by mechanically ruling; (A5) laminar holographically recorded and ion etched groove profile. (B) and (C) are AFM scans of surface of peeled patterned (aluminium) foils of (B) a CD-R with stripe periodicity 1.5 μm, stripe width 750 nm, and stripe height 130 nm; and (C) a DVD-R with stripe periodicity 800 nm, stripe width about 400 nm, and stripe height about 70 nm.

FIG. 5 are photographs showing examples of diffraction gratings. (A) Holographic diffraction grating film of size 6″×12″ sheet held against light; and (B) CD-R with light shined upon.

FIG. 6 is a graph showing zeta potential of the top surface of processed optical discs to demonstrate that electrical charge is present on the surface. Both CD-R and DVD-R had overall negative potential on their surfaces.

FIG. 7(A) is a series of images showing water contact angles (WCA) of surfaces of (i) and (ii) CD-R; (iii) and (iv) DVD-R; and (v) and (vi) 12700 gratings before and after oxygen plasma treatment. From the figure, it may be observed that the water contact angles decreased after plasma treatment. FIG. 7(B) is a graph depicting quantitative analysis of water contact angles of CD-R, DVD-R, 12700 gratings and 25400 gratings, as well as their respective water contact angles after oxygen plasma treatment. The water contact angles for the CD-R and DVD-R samples decreased from about 90° to about 60° after plasma treatment. For the gratings samples, the decrease in water contact angle was more drastic. For 12700 gratings, WCA decreased to about 10° and for 25400 gratings, WCA decreased to about 33°.

FIG. 8 is a SEM image of a CD-R sample with gelatin coating. From the image, it may be seen that there is presence of gelatin nanospheres on the ridges and inside the grooves, thereby demonstrating that the surfaces of the gratings may be coated with extra-cellular matrix (ECM) proteins, and the ECM proteins do not mask the micro grooves or nano grooves present on the CD-R surface.

FIG. 9 are Brighfield images showing 3T3 cells cultured for 24 hrs on CD-R gratings on a dish. Cells were cultivated on an ungrooved side of the CD-R and used as control. As seen from the figure, cells on the control in (A) are randomly distributed on the surface and did not align. On the other hand, cells on the grooved side as shown in (B) aligned along the grating direction. Insets in the images indicate the grating direction. Images were also taken at various magnifications of (C) 10×; (D) 20×; and (E) 40× to demonstrate the aligned and stretched morphology of fibroblasts. At 40×, grooves may be observed in the image.

FIG. 10 depicts a series of results from measurements taken to quantify alignment of HL-1 cells growing on CD-R gratings on a dish. (A) F-actin staining shows clearly the cellular cytoskeleton of HL-1 cells cultured on CD-R surface for 24 hrs; (B) first step of the measurement algorithm involved identifying the edge (map) of the cells; (C) actual image of the cells overlaid with the edge map obtained in (B); (D) selected cells (depicted in red and as marked in the figure), in which the edge map co-localized with the actual cell image, for alignment calculation; (E) histogram plot of the direction result showing that majority of the cells preferentially align at an angle of about -50 ⁰; and (F) the results in (E) presented in a Rose-plot format.

FIG. 11(A) (i) and (ii) are images showing F-actin staining for H9C2 cells cultured on holographic gratings (gratings on a dish) having pitch of 1 μm for 2 days. The alignment was prominent in H9C2 cells because of their large size. The circled portions denote DAPI staining (in blue and as marked in the figure) for nuclei of some of the cells. Scale bar in the figures denote a length of 30 μm. (B) are images of F-actin staining showing cell attachment and alignment on the gratings of 2 micron pitch (500 lines/mm). All the three cell types of cardiac origin (HL-1, H9C2 and primary cardiomyocytes) aligned on the grooved surface of the grating. The alignment was prominent in H9C2 cells because of their larger size and fewer numbers compared to the primary or HL-1 cells. The circled portions denote DAPI staining (in blue and as marked in the figure) for nuclei of some of the cells.

FIG. 12 are graphs showing (A) Alamar blue assays for 3T3 fibroblasts proliferation on (i) gratings on a dish and (ii) control (ungrooved). Proliferation rate of cells was similar for the grooved and ungrooved surfaces. Data are represented as average±standard deviation of 4 samples. (B) Alamar blue assay for H9C2 cell proliferation on (i) gratings (2-micron pitch) in relation to growth on ungrooved surface on (ii) control and (iii) glass cover slip. Growth rate of cells was similar on the grooved surface of the gratings, the ungrooved surface and the glass coverslips. The results demonstrate that cell growth is normal on the grooved surface.

FIGS. 13(A) and (B) are images showing cell-cell interaction demonstrated by N-Cadherin (cell adhesion protein) staining of rat primary cardiomyocyte (grown on gratings). N-Cadherin, which is a calcium dependent cell-cell interaction protein, is aligned along the direction of the grooves of the gratings as shown. Uniform arrangement of the cell adhesion protein N-Cadherin enhances directional communication between the cells, and indicates an improved physiological state of the cardiomyocytes.

FIG. 14(A) to (F) are images showing cell differentiation on DVD-R surface using C2C12 cells stained with (A) and (D) myosin heavy chain protein (red); (B) and (E) F-actin protein (green); and (C) and (F) merged images of myosin heavy chain and F-actin proteins. Alignment of the cells on the grooved surface is shown clearly on the top panel (A)-(C). In addition, the cells on the grooved surface of the DVD had fused to form longer myotubes, indicating a more mature phenotype. In contrast, cells on the ungrooved surface (D)-(F) are randomly oriented. Furthermore, the samples indicated that there are fewer cells expressing the MHC, and that the cells have shorter myotubes.

FIG. 15(A) to (F) are images showing cell differentiation on CD-R surface using C2C12 cells stained with (A) and (D) myosin heavy chain protein (red); (B) and (E) F-actin protein (green); and (C) and (F) merged images of myosin heavy chain and F-actin proteins. The alignment of the cells on the grooved surface is clearly depicted on the top panel (A)-(C). In contrast, cells on the ungrooved surface as shown in (D)-(F) are randomly oriented, and only a few cells show the expression of differentiation markers.

FIG. 16(A) to (F) are images showing cell differentiation demonstrated on gratings (2 micron pitch) using C2C12 cells stained with (A) and (D) myosin heavy chain protein (red); (B) and (E) F-actin protein (green); and (C) and (F) merged images of myosin heavy chain and F-actin proteins. The alignment of the C2C12 cells on the grooved surface is clearly depicted on the top panel (A)-(C). In contrast, cells on the ungrooved surface (D)-(F) are randomly oriented and only few cells show the expression of differentiation markers.

FIG. 17 is a graph showing differentiation index [Ratio of the red signal (myosin heavy chain) to the green signal (F-actin)]. Quantitative analysis of average differentiation was carried out across various surfaces (mentioned in FIG. 14-16). From the graph, it may be seen that the cells depict maximum differentiation for grooved surface of DVD-R, whereas differentiation is minimum for the ungrooved surface of the gratings.

FIG. 18 is a flow chart depicting general methods to make a device for cell or tissue culture (“gratings on a dish”) according to various embodiments. Embodiments relating to the use of polycarbonate component layer (for example, polycarbonate component layer in a CD-R without the overlying aluminium and lacquer layers) comprising pre-grooves for cell culture are not shown in the flow chart.

### DETAILED DESCRIPTION OF THE INVENTION

Various embodiments of the invention relate to the use of a commercially available optical medium, which may be further processed and made suitable, for cell culture and cell alignment. Due to their grated surface, commercially available optical media, such as CD-Rs, DVD-Rs and holographic gratings, may directly be utilized to culture cells and for cell alignment. In so doing, issues relating to use of conventional patterning processes, such as electron beam lithography and photolithography, which require costly and tedious clean-room equipments and protocols to create large area grooved substrates, are eliminated.

Accordingly, in a first aspect, the invention refers to a method of culturing cells or tissues. The method includes providing a support, wherein the support is an optical medium with a patterned surface. As used herein, the term “optical medium” refers to a component having a patterned surface used in photonics or applications involving electromagnetic waves. In various embodiments, the patterned surface of the optical medium is able to interact with electromagnetic waves, such as light, for example, by changing the direction of the waves, or by splitting and diffracting light into several beams travelling in different directions. Examples of optical media include, but are not limited to, compact discs (CDs), compact disc recordables (CD-Rs), compact disc rewritables (CD-RWs), digital versatile disc (DVDs), digital versatile disc recordables (DVD-Rs, DVD+Rs), digital versatile disc rewritables (DVD-RWs, DVD+RWs), blu-ray discs (BDs), blu-ray disc recordables (BD-Rs), blu-ray disc rewritables (BD-Res), laser discs (LDs), mini discs (MDs), hybrid discs, and holographic gratings.

In various embodiments, the patterned surface of the optical medium comprises or consists of a grating. In some embodiments, the grating is a diffraction grating. As used herein, the term “diffraction grating” refers to an optical component having a periodic structure, which is able to split and diffract light into several beams travelling in various directions, wherein the grating acts as a dispersive element. The direction of the light beams may depend on factors, such as physical dimension, such as width or spacing, of the grating and wavelength of the incident light beam.

Diffraction gratings may be used in a variety of applications such as spectrometers, which are tools used to measure different properties of a light source; monochromators, which are tools used to consolidate light wavelengths; holograms, which refer to three-dimensional images embedded in a flat surface; fiber-optic communications, which are based on the working principle that by splitting the data transmitted into varying wavelengths, multiple data streams can be send over the same strand of wire; optical storage media, such as DVDs, CDs, and BDs; lasers, which are commonly used in devices such as the DVD player or gaming console; and light polarization devices.

Diffraction gratings may assume various sizes and shapes depending on the type of optical medium. FIG. 4 depicts examples of diffraction gratings with different groove shapes. Advantageously, the availability of diffraction gratings having different aspect ratios and different groove designs such as sinusoidal, pseudosinusoidal, concave, and ellipsoids, in the form of different optical media provide opportunities for more in-depth cell and mechanobiology related studies.

Depending on the type of optical medium, diffraction gratings may be present as different forms on the optical medium. For holographic gratings, for example, the diffraction gratings may be present as micro or nano grooves on the surface of a substrate. The terms “micro” and “nano” refer respectively to a measure of the physical dimension that may be used to define the size of the grooves, for example, at least one of length, height and width, in units ranging from several nanometers (for nano grooves) to several micrometers (for micro grooves). For example, a micro groove generally refers to a groove having at least one physical dimension (such as length, height or width) that is more than about 100 nm, such as about 200 nm, 300 nm, 400 nm, 500 nm, 600 nm, 700 nm, 800 nm, 900 nm, 1 μm, 2 μm, 5 μm or 10 μm. A nano groove, on the other hand, generally refers to a groove having at least one physical dimension that is less than or equal to about 100 nm, such as about 1 nm, 5 nm, 10 nm, 20 nm, 30 nm, 40 nm, 50 nm, 60 nm, 70 nm, 80 nm, 90 nm, or 100 nm.

For optical discs such as CD-R and DVD-R for example, the diffraction gratings may be present as grooves on the recording layer of the CD-R or DVD-R, which may be covered with one or more other layers. Such grooves are also termed “pre-grooves”, which refers to grooves that are formed in advance on the recording layer of a recordable CD, for example, grooves formed on a polycarbonate component layer in a CD-R, prior to formation of any overlying layers on the polycarbonate component layer. The pre-grooves may, for example, be used for tracking purposes to ensure that the laser head can write the data into the groove along the rails; for addressing purposes to ensure the recorder to write in data to the accurate place; and/or for speed to control or guide the driver to rotate at a certain speed and in a certain way.

The micro or nano grooves comprised in the diffraction gratings of various optical media may simulate biomechanical properties of extracellular matrix materials around a cell. Using CD-R as example, a CD-R generally comprises a layer of polycarbonate, an aluminium layer formed on the polycarbonate surface, and a lacquer layer coated on the aluminium layer. The polycarbonate layer may constitute the recording layer of the CD-R. Typically, patterns are drilled in the form of spiral tracks or pre-grooves on a surface of the polycarbonate. The patterned surface of the polycarbonate may be used to form a support upon which cells are cultivated. A DVD-R typically comprises two joined layers of polycarbonate which may be dislodged by applying force to separate the two layers. Either one or both polycarbonate layers may contain a patterned surface that may be used as a support for cell culture.

The method of culturing cells or tissues according to various embodiments of the invention may be used to grow or culture cells or tissues that are at least substantially aligned with the patterned surface of the optical medium. As used herein, the term “align” refers to growth and orienting of cells in one or more general directions defined by the patterns present on the optical medium such that the cells arrange or fall into position according to the patterns on the surface of the optical medium. For example, in embodiments in which the optical medium comprises or consists essentially of grooves which are arranged at least substantially parallel to one another, the cells may orient themselves such that the long axis of the cells are at least substantially parallel to the grooves.

Use of diffraction gratings, which are by themselves commercially and readily available, as cell-culture substrates means that substrates containing such surface patterns are ready for use, thereby negating the need for a user to fabricate the patterned substrate. This translates into a faster and easier route to culture cells or tissues on a patterned surface. In various embodiments, the cells or tissues that are grown on the patterned surface of the optical medium are substantially aligned with the diffraction gratings. The use of a patterned surface of an optical medium as support for cell culture alleviates the need to invest in costly clean room equipment and/or engage in lengthy protocols to fabricate the patterned substrate. This is important, particularly for small laboratories or startup companies which may not have access to the capital intensive equipment. Accordingly, by using methods according to various embodiments of the invention, these entities are not barred from elaborate biological studies and research simply because they do not have access to the expensive equipment.

In addition, the low prices of commercially available optical media (for example, retail price of CD-R/DVD-R is less than S$1 per piece; retail price of holographic grating is about S$8 for a 6″×12″ sheet size) translates into substantial cost savings, for the end user. Given the larger size of the patterned surface on an optical medium, compared to the much smaller patterned areas typically fabricated using methods such as electron beam lithography and laser radiation, the optical medium may be cut or diced into smaller pieces for use, thereby translating into even greater cost savings for the user.

Other advantages include ease of imaging cells growth, in view that the patterned surface of optical media such as CD-R is generally formed on polymer such as polycarbonate, which is at least substantially transparent. The method according to the first aspect is also environmentally friendly, as used CD-Rs or DVD-Rs, which are in most cases discarded as waste, may be re-used or recycled for this purpose.

In various embodiments, the optical medium comprises or consists of a biocompatible material. As used herein, the term “biocompatible” refers to substances that are not toxic to cells. For example, a substance may be considered to be biocompatible if, upon contact of the substance with cells, there is less than or equal to about 10%, about 5%, or less than about 5% cell death.

In various embodiments, the optical medium comprises or consists of polymer, glass, silicon, metal, alloys, metal oxides, metal carbides, metal fluorides, or mixtures thereof. In some embodiments, the optical medium comprises a polymer. For example, the optical medium may comprise or consist of a polymer selected from the group selected from polycarbonate, polyester, polystyrene, poly(methyl methacrylate), polyurethane, polyacrylic acid, derivatives thereof and copolymers thereof. In various embodiments, the optical medium comprises or consists essentially of polycarbonate.

To render the patterned surface suitable for cell growth, in particular, in cases where recycled optical media is used, the method according to the first aspect may include treating the patterned surface prior to contacting the support with cells and a culture medium, the culture medium optionally being nutrient-containing. For example, in case of optical discs, the patterned surface may contain other layers of materials such as chemicals and dyes, and may be non-sterile. In such cases, the chemicals and dyes on the patterned media have to be removed as they may be toxic to living cells.

In various embodiments, treating the patterned surface comprises contacting the patterned surface with a solvent to remove organic material from the patterned surface. For example, by using a solvent, the dye or paint on the surface of the CD-R may be removed. Generally, any suitable solvent may be used. For example, the solvent may be a non-polar solvent, a polar solvent, a mixture of two or more non-polar solvents, or a mixture of two or more polar solvents.

A non-polar solvent refers to a solvent that has no measurable dipole. Specifically, it refers to a solvent having a dielectric constant of less than 15, less than 10 or between about 5 to about 10. Examples of a non-polar solvent include, but are not limited to, alkanes such as butane, hexane, octane, cyclohexane, aromatic compounds such as benzene and toluene, diethyl ether, chloroform and 1,4-dioxane.

A polar solvent, on the other hand, refers to a solvent that exhibits polar forces on solutes which can happen as a result of high dipole moment, wide separation of charges or tight association such as water, alcohols and acids. The solvents typically have a measurable dipole, and have a typical dielectric constant of at least 15, at least about 20 or between 20 to about 30. Examples of a polar solvent include, but are not limited to, ethyl acetate, tetrahydrofuran (THF), dichloromethane (DCM), N-methyl-2-pyrrolidone (NMP), ketones such as acetone, methyl ethyl ketone and methyl isobutyl ketone; acetonitrile, dimethylformamide (DMF), dimenthyl sulfoxide (DMSO), alcohols such as methanol, ethanol, propanol, isopropyl alcohol, n-butyl alcohol, sec-butyl alcohol, t-butyl alcohol, isobutyl alcohol and diacetone alcohol; glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; formic acid, acetic acid, an aqueous solution, water, and mixtures thereof. In various embodiments, the solvent comprises or consists essentially of methanol.

In various embodiments, treating the patterned surface comprises contacting the patterned surface with a disinfectant or autoclaving to sterilize the patterned surface. As used herein, the term “disinfectant” refers to an agent that provides antimicrobial or microbiocidal activity, so as to destroy, neutralize, or otherwise interfere with the growth or survival of microorganism. In various embodiments, the disinfectant kills at least substantially all microorganisms or microbes that are residing or present on a surface when the disinfectant is applied.

The disinfectant may be in the form of a chemical reagent that is applied to the patterned surface, or in the form of an electromagnetic radiation such as ultra-violet radiation or gamma radiation that is irradiated on the patterned surface. The patterned surface may also be placed in an autoclave, which is a device that provides saturated steam at high pressures, to sterilize the patterned surface. One or more types of disinfecting method may be used in combination i.e. concurrently. Examples of chemical reagents that may be used as the disinfectant include, but are not limited to, ethanol, isopropanol, chlorhexidine gluconate, povidoneiodine, ethylene oxide (oxirane) or mixtures thereof. In some embodiments, the disinfectant comprises or consists essentially of ethanol. The support may, for example, be immersed in a solution comprising the chemical reagent. In other embodiments, the support may be disinfected by spray coating or dip coating into a solution comprising the chemical reagent. Ultraviolet radiation may also be used to disinfect a patterned surface of the optical medium. The patterned surface may be irradiated for a time period that is sufficient to sterilize the surface.

The method according to the first aspect may include treatment steps to render the patterned surface hydrophilic. By modifying the hydrophilicity of the patterned surface, subsequent attachment of cells on the patterned surface may be facilitated. For example, a layer of hydrophilic compounds may be deposited on the patterned surface to render the patterned surface hydrophilic. In various embodiments, treating the patterned surface to render the patterned surface hydrophilic comprises treating the patterned surface with an ionized oxygen-containing plasma.

The method according to the first aspect may include exposing a patterned surface of an optical medium. As mentioned above, optical media such as CD-R or DVD-R may comprise one or more layers of material coated on the patterned surface. For example, a CD-R generally comprises a layer of polycarbonate, an aluminium layer formed on the polycarbonate, and a lacquer layer formed on the aluminium layer. The aluminum layer may function as a reflective layer to allow reflection of light from the CD-R for reading of data and/or for data retrieval purposes. The lacquer layer may function as a protective layer to protect the underlying aluminium layer and patterned polycarbonate. Accordingly, exposing the patterned surface on the polycarbonate layer may include physically removing layers of material coated on the patterned surface. For example, by using a tweezer, the polycarbonate layer of a CD-R may be separated from the aluminum layer and the lacquer layer.

Methods of treating the patterned surface to render the patterned surface suitable for cell growth may include one or more of the above-mentioned treatment procedures. The choice of treatment may depend on the type of optical medium used. In some embodiments, the optical medium is not treated. For example, in embodiments whereby only the underlying polycarbonate patterned layer of a CD-R is provided, or in cases where holographic gratings are used, the optical media may be used as received, and no treatment may be needed to render the patterned surface suitable for cell growth.

In various embodiments, the patterned surface of the optical medium comprises or consists essentially of grooves which are arranged at least substantially parallel to one another. In some embodiments, the patterned surface of the optical medium comprises or consists essentially of pre-grooves.

Typically, while writing data on recordable optical media such as a CD-R or a DVD-R for example, pits are written along the tracks of the polycarbonate recording layer, thereby providing a more complex surface pattern. Similarly, CDs and DVDs which are molded from a master transfer the digital data in the form of pits and lands onto the polycarbonate surface. As such, surface topography of used CD-R/DVD-R and conventional CDs/DVDs are different from that of blank CD-Rs and DVD-Rs, which contain spiral pre-grooves which are typically formed or molded by a metal stamper. Presence of the pre-grooves on blank CD-Rs and DVD-Rs for example, are particularly advantageous as they allow cell alignment along the pre-grooves, whereby cells are allowed to grow uniformly along the grooves. In contrast thereto, the pits present on used CD-Rs/DVD-Rs and CDs/DVDs may disrupt cell growth and/or alignment of the cells. Accordingly, in various embodiments, the optical medium is a blank optical medium or a blank optical disc. In some embodiments, the optical medium is selected from the group consisting of CD-Rs, CD-RWs, DVD-Rs, DVD-RWs, DVD+Rs, DVD+RWs, BD-Rs, BD-REs, laser disc, mini disc, hybrid disc, and holographic gratings.

The method includes contacting the support with cells and a culture medium. In various embodiments, the cells comprise or consist essentially of mammalian cells.

A mammalian cell refers to any cell that is derived from a mammal. Examples of mammals include, but are not limited to, a rat, a mouse, a rabbit, a guinea pig, a squirrel, a hamster, a hedgehog, a platypus, an American pika, an armadillo, a dog, a lemur, a goat, a pig, an opossum, a horse, an elephant, a bat, a woodchuck, an orang-utan, a rhesus monkey, a woolly monkey, a macaque, a chimpanzee, a tamarin (saguinus oedipus), a marmoset or a human. The cells may for instance be cells of a tissue, such as an organ or a portion thereof. A mammalian cell may include a mammalian cell line. In one embodiment, the mammalian cell may be a human cell. Examples of a human cell include, but are not limited to, an osteogenic cell, a fibroblast, an epidermal cell, an adipocyte, a neural cell, an endothelial cell, an epithelial cell, a keratinocyte, a hepatocyte, a myocyte, a cardiomyocyte, a cell from joint ligament, a cell from the nucleus pulposis, a HEK 293 cell and PER.C6® cell.

An osteogenic cell refers to an osteoblast or a progenitor osteoblast cell, which gives rise to a bone tissue. A fibroblast is a spindle shaped cell which can rapidly replicate and synthesize a fibrous matrix composed of a variety of extracellular matrix molecules including Type I Collagen, and which can be found in skin. An epidermal cell refers to a cell of the epidermis, wherein the epidermis is the outer layer of skin and is composed of four types of cells, i.e. keratinocyte, melanocyte, Langerhans cell, and Merkel cell. The term “adipocyte” refers to a cell existing in or derived from fat tissue which is terminally differentiated. It is also known as a lipocyte or fat cell, and specializes in storing energy as fat. In their differentiated state, adipocytes assume a rounded morphology associated with cytoskeletal changes and loss of mobility. Neural cells refer to cells of the nervous system and in particular of the brain. Examples of neural cells include, but are not limited to, neurones, astrocytes and oligodendrocytes. Endothelial cells refer to a thin, flattened cell, of which a layer of the cells lines the inside surfaces of body cavities, blood vessels and lymph vessels, making up the endothelium. The term “epithelial cell” refers to a cuboidal-shaped, nucleated cell which is generally located on the surface of a tissue. A layer of epithelial cells generally functions to provide a protective lining and/or surface that may also be involved in transport processes. The term “keratinocyte” refers to skin cells having the capability to produce keratin, including for example, cells known as basal cells, prickle cells, spinous cells, and granular cells. A hepatocyte is a cell that constitutes the main functional cells of the liver, and can constitute 60 to 80% of the mass of a liver tissue. Hepatocytes perform critical metabolic, endocrine, and secretory functions, which includes the synthesis of carbohydrates, cholesterol and bile salts, to name a few. Stem cells refer to cells having self-replicating ability and also the ability to differentiate into at least two cells, and can be divided into totipotent stem cells, pluripotent stem cells and multipotent stem cells. Myocte refers to a differentiated, post-mitotic, muscle cell that has not undergone fusion and represents a transient cell type under most conditions. Cell from joint ligament can comprise a chondrocyte or a fibroblast from the articular ligament, peritoneal ligament or fetal remnant ligant, which are important as ligaments connect a bone to another bone to form a joint which is required for mobility. Cells from the nucleus pulposis have chondrocyte-like features. In an adult human, the cells of the nucleus pulposis obtain nutrients and eliminate waste by diffusion through blood vessels in the endplates of the vertebrate adjacent to the intervertebral discs. A HEK 293 cell is a human embryonic kidney cell line, and PER.C6® cell is a human retina cell line.

Any type of cell may be added to the support for culturing, including cells of the muscular and skeletal systems, such as chondrocytes, fibroblasts, muscle cells, and osteocytes, parenchymal cells such as hepatocytes, pancreatic cells (including Islet cells), cells of intestinal origin, and other cells such as nerve cells and skin cells, either as obtained from donors, from established cell culture lines, or even before or after genetic engineering. Pieces of tissue may also be used, which may provide a number of different cell types in the same structure.

In various embodiments, the cells are selected from the group consisting of organ cells, muscle cells, nerve cells, stem cells, epithelial cells, connective tissue cells, cancerous cells (cell lines), and combinations thereof. In more specific embodiments, the cells are selected from the group consisting of human embryonic stem cells (hECs), human mesenchymal stem cells (hMSCs), keratinocytes, tenocytes, ligament cells, cardiomyocytes, osteoblasts, fibroblasts, myoblasts, endothelial cells, and combinations thereof. In even more specific embodiments, the cells are selected from the group consisting of Chinese Hamster Ovary (CHO) cells, COS cells, HL-1 cells, H9C2 cells, 3T3 cells, C2C12 cells, PC12 cells, NIH3T3 cells, HeLa cells, and combinations thereof.

Generally, any culture medium that allows cells to grow and/or proliferate may be used. In various embodiments, the culture medium is a nutrient-containing culture medium.

For example, the culture medium may comprise or consist of an aqueous medium for culturing cells, such as one of the well known cell culture media (“growth media”) available in the art, e.g. LB medium; a monosaccharide containing liquid—possibly including e.g. Hank's Salts; Eagle's minimal essential medium (including e.g. Dulbecco's Modified Eagle Medium (DMEM)); RPMI (Roswell Park Memorial Institute) medium; HyClone medium; Ham's tissue culture medium; Chee's medium; YM Broth; or Murashige and Skoog medium, to name a few, or blood.

The method includes culturing the cells under suitable cultivation conditions. Various cell types may attach, proliferate, and align with the gratings. As mentioned above, the cells may grow along the general direction defined by the patterns present on the optical medium to form cells or tissues that are at least substantially aligned with the patterned surface of the optical medium. The gratings may be coated with chemical ligands and extracellular matrix proteins (ECM) such as, but not limited to, gelatin, fibronectin, laminin, collagen I/IV, Poly-L-Lysine, Poly-D-Lysine, or mixtures thereof to modulate cell attachment, proliferation and function. The cells may be allowed to grow or to proliferate for a time period, in which the cells may grow to form colonies. Generally, the time for proliferation may range from a few hours or days to a few weeks, such as about 1 day to about 4 weeks, or about 1 day to about 2 weeks, or about 1 day to about 1 week. The time for proliferation may also depend on the cultivation conditions for the cells. Parameters of the cultivation condition may include, for example, temperature, pH, amount of water, pressure, nutrients present, and type of cell. For example, it is well known that eukaryotic mammalian cells grow much slower in general than for example prokaryotic bacterial cells. Cultivation conditions of cells are known in the art and can therefore be adapted by a person skilled in the art depending on the desired cell type and application.

Generally, different living cell species may be cultivated by a method of the invention. The cell species may group together to form tissues. The term “tissue” refers to a structure formed by related cells joined together, wherein the cells work together to accomplish specific functions. Different types of tissues may also be arranged together to form organs. An organ refers to a differentiated structure of an organism composed of various cells or tissues and adapted for a specific function. Therefore, one or more species of living cells can be added and cultivated to form a specific organ or part thereof.

For example, the heart which is an organ contains muscle tissue that contracts to pump blood, fibrous tissue that makes up the heart valves and special cells that maintain the rate and rhythm of heartbeats. As another example, one or more species of living cells can be added and cultivated to form a skin specimen for transplantation purposes.

The aligned cells formed using methods of the invention may be used for various tissue engineering applications, such as growing sheets of vascular smooth muscle cells, and remodelling and maturation of isolated cardiomyocytes. One example is human mesenchymal stem cells, which have been shown to differentiate into neuronal lineage after being cultured on nano-gratings with growth factors, or undergo osteogenic, adipogenic, and chondrogenic differentiation on aligned collagen. Another potential application area relates to human embryonic stem cells, as they may be directly differentiated to form selective neurons on nanoscale groove patterns without using growth factors. Yet another potential application area relates to the heart, whereby the heart in vivo is an anisotropic organ and the cardiomyocytes are aligned in the heart because of the arrangement of the collagen fibers. By replicating this anisotropic structure in vitro, aligned contraction, which is essential for heart function, may be produced. In this respect, methods according to the first aspect are advantageous over state of the art methods as expensive growth factors reagents are not required for the aligned cell growth. Furthermore, given that the reagents are not used, this alleviates the need to precisely control the exact concentration of those reagents for efficient differentiation of cells.

In a second aspect, the invention refers to a device for cell or tissue culture comprising an optical medium, wherein a patterned surface of the optical medium forms a support upon which cells are cultivated.

In various embodiments, the patterned surface of the optical medium comprises or consists of a grating. For example, the grating may be a diffraction grating. Examples of optical media that may be used have already been described herein.

In its various forms, the diffraction grating may comprise alternating protrusions and grooves, wherein the alternating protrusions and grooves are arranged at least substantially parallel to one another. For example, the protrusion may have a height of between about 70 nm to about 300 nm, such as between about 100 nm to about 200 nm, or between about 200 nm to about 300 nm. In various embodiments, the protrusion or groove may each have a width of between about 400 nm to about 1 μm, such as between 400 nm to about 600 nm, between about 500 nm to about 800 nm, or between about 600 nm to about 1 μm. In some embodiments, the diffraction gratings have a period of between about 800 nm to about 2 μm, such as between about 800 nm to about 1.5 μm, or between about 1 μm to about 2 μm. Depending on the cells or tissues to be cultivated, for example, it is not necessary that each protrusion or groove that is present on the optical medium have the same dimension or are spaced equally apart on the optical medium. Examples of cells that may be used have already been described above.

In view of the above, using CD-R as an example, the width and depth of each line or groove on the spiral track of a CD-R are about 750 nm and about 130 nm respectively, while the separation distance between the two lines is about 1.5 μm. Since the diameter of a typical CD-R is about 120 mm, i.e. much larger compared to the separation distance between two lines or grooves on the CD-R, when viewed under an optical microscope, the grooves appear parallel with almost infinite radii of curvatures. More importantly, when the CD-R is cut into smaller pieces for use, the spiral grooves present on the CD-R translates into near parallel or parallel grooves on the smaller pieces. The aluminum coating on the polycarbonate surface possesses similar patterns, which also appear as parallel lines under a microscope.

In case of a DVD-R, DVD-R typically comprises two joined layers of polycarbonate which may be separated, for example, by using a tweezer and applying force to pull them apart. The usual width and depth of each line the spiral track of a DVD-R are approximately 400 nm and 70 nm respectively, while the separation distance between the two lines is about 800 nm.

In its use as a support for cell culture, the patterned surface of the optical medium may form a base plate upon which cells are cultivated in the device. However, it is not necessary that the patterned surface of the optical medium form the base place of the device. The patterned surface may also act as a support for culturing of cells in a different orientation, such as a vertical side plate, or placed with the patterned surface facing downwards (along the direction of gravity). Such configurations may be adopted, for example, in carrying out investigations relating to the effect of gravity on cell growth, for example.

In various embodiments, the device for cell or tissue culture may be a cell culture dish or a well plate or a flask. As mentioned above, the optical medium may be dimensionalized by cutting into smaller pieces and incorporated into the cell culture dishes or wells or flasks.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### EXAMPLE 1

### Processing of the Diffraction Gratings for Cell Culture

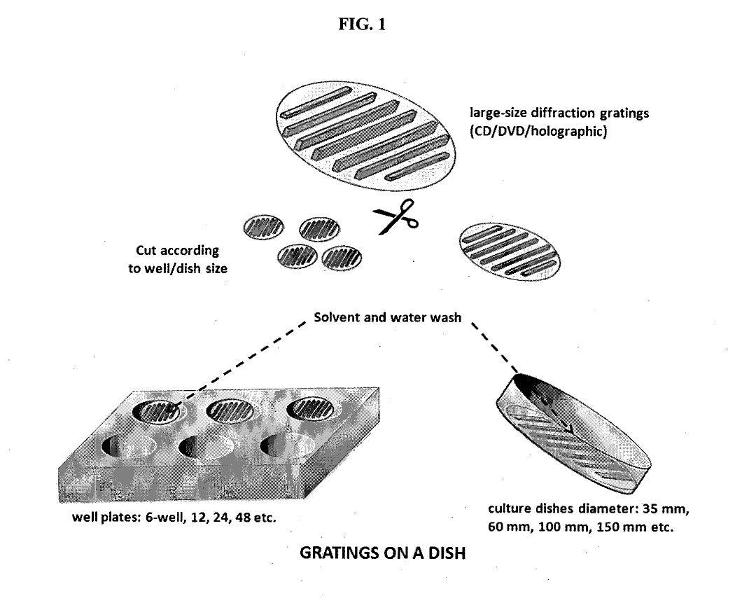

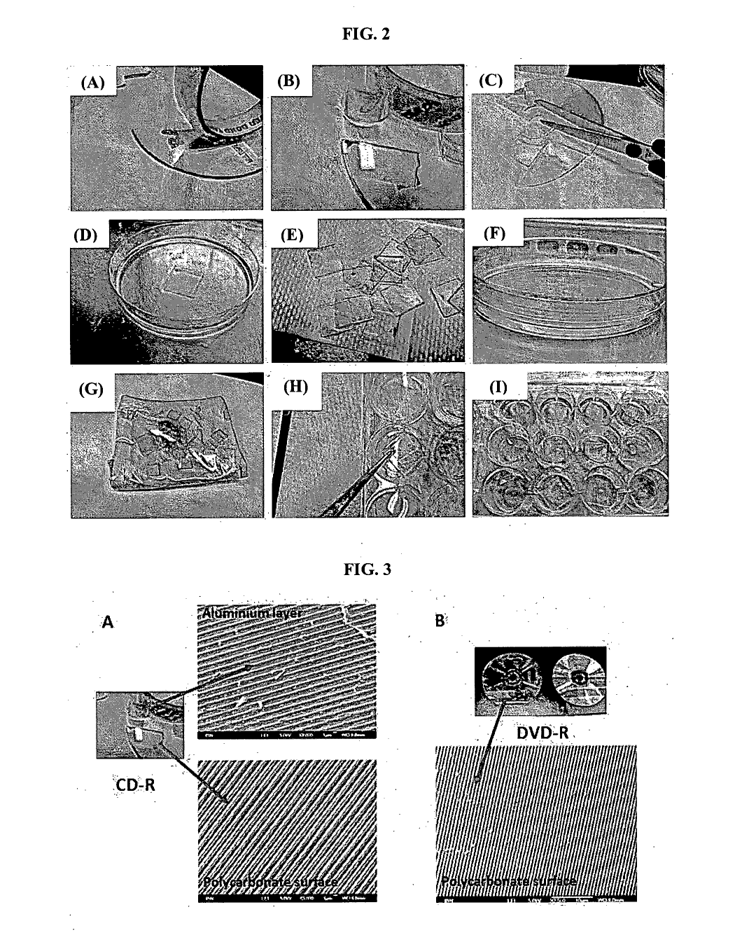

FIG. 1 is a schematic diagram showing a method to fabricate a device for cell or tissue culture (“gratings on a dish”) according to various embodiments. FIG. 2 is a series of photographs depicting processing of the substrate surface of CD-R to form gratings on a dish according to various embodiments.

Holographic gratings (6″×12″) were first punched to obtain 13 mm round films for 24-well plates. For CD-Rs, the label, acrylic and aluminum layers over the polycarbonate layer were easily, peeled off using an adhesive tape. For DVD-Rs, a tweezer was used to carefully dislodge the junction to expose two polycarbonate surfaces. The bottom polycarbonate surface of DVD-R was processed further. The exposed polycarbonate surfaces of CD-Rs/DVD-Rs were cut into pieces (1 cm×1 cm) using scissors, so that they may fit in 24-well plates. Small dots were punched on the grooved surface for subsequent identification.

The gratings and CD-R/DVD-R pieces were treated with absolute methanol for 2 hrs followed by sonication for 30 mins. The pieces were then rinsed with distilled (DI) water to remove any chemicals/dust particles on the surface. The methanol and/or DI water was used to remove the organic dyes in case of the optical discs. The pieces were then treated with 70% ethanol for 2 hrs, followed by sonication for 30 mins and rinsing with DI water to make the surface sterile. For absolute sterility, autoclaving of the diffraction gratings was carried out at 105° C. for 21 mins to make them ready for cell culture. The gratings pieces were then placed in a well plate to complete “gratings on a dish” device.

### EXAMPLE 2

### Atomic Force Microscope (AFM) and Scanning Electron Microscopy (SEM) Characterization of the Surfaces of the Diffraction Gratings

SEM and AFM samples were prepared by cutting appropriate sizes of the diffraction gratings. For SEM, the samples were viewed with a JSM 5600 scanning electron microscope (Jeol, Japan) at 5 kV. Prior to imaging, the gratings were sputter-coated with platinum for 60 s. For CD-R samples with 0.1% gelatin, the protein was fixed by glutraldehyde and serially dehydrated with ethanol before SEM was done. Atomic force microscope, DI Nanoscope Dimension 3100 (Digital Instruments, USA) was used in tapping mode to determine the features on the holographic gratings.

### EXAMPLE 3

### Zeta Potential Measurement of the Surface

The zeta potentials of the diffraction gratings were measured by using an electro kinetic analyzer (EKA 1.00, Anton-Paar GmbH, Graz, Austria) equipped with a plated sample cell. The membranes were cut into 2 cm×1 cm pieces. The measurements were conducted at 25° C. in 1 mM of potassium chloride (KCl) solution at pH 6.8. In each case, measurements were performed with three replicas.

### EXAMPLE 4

### Water Contact Angle Measurement of the Surface

Five microliter of DI water was pipetted on the gratings surface at normal velocity of dispension. Water contact angles were measured with a goniometer (Contact Angle System OCA 30, Data Physics Instruments GmbH, Germany) using the SCA20 software.

### EXAMPLE 5

### Coating the Surface with Extracellular Matrix Proteins

The gratings and CD-R/DVD-R were coated with gelatin (0.1%) after placing them in the 24-wells. The incubation time was kept at 12 hrs.

### EXAMPLE 6

### Mammalian Cell Culture in “Gratings on a Dish”

Various types of cells; HL-1 (cell line from mouse atrial cardiomyocyte tumor lineage), H9C2 (cell line from embryonic rat ventricle) and 3T3 (cell lines from primary mouse embryonic fibroblast cells) were used in the experiments to demonstrate attachment and alignment of the cells on the diffraction gratings inside the 24-well plates. Firstly, cells were cultured on tissue culture plates with DMEM media and on reaching confluency, were trypsinized and then seeded on the gratings. Primary cardiomyocytes was also cultured using DMEM media, but was used only once after isolation from the rats (i.e. not passaged).

### EXAMPLE 7

### Cell culture of NIH/3T3 and H9C2 Cells (Adapted from ATCC)

### Example 7.1

### Handling Procedure for Frozen Cells

The culture protocol was adapted from various ATCC protocols as indicated herein which are known to a person skilled in the art as standard ways of culturing mammalian cells. Cell line designation H9c2(2-1) was cultured using protocol adapted from ATCC Catalog No. CRL-1446. Cell line designation 3T3-Swiss albino was cultured using protocol adapted from ATCC Catalog No. CCL-92.

To ensure the highest level of viability, the vial was thawed and the culture was initiated as soon as possible upon receipt. In case continued storage of the frozen culture was required, it was stored in liquid nitrogen at vapor phase.

The vial was thawed by gentle agitation in a 37° C. water bath for approximately 2 minutes. To reduce the possibility of contamination, the O-ring and cap were kept out of water.

The vial was removed from the water bath as soon as the contents were thawed, and decontaminated by dipping in or spraying with 70% ethanol.

The vial contents were transferred to a centrifuge tube containing 9.0 ml complete growth medium and spun at approximately 125×g for 5 minutes.

The cell pellet was resuspended with the recommended complete growth medium (specific batch information for the culture recommended dilution ratio), and dispensed into a 25 cm² or a 75 cm² culture flask. Prior to addition of the vial contents, the culture vessels containing the complete growth medium were placed into the incubator for at least 15 minutes to allow the medium to reach its normal pH (7.0 to 7.6).

The culture was incubated at 37° C. in a suitable incubator. A 5% carbon dioxide (CO₂) in air atmosphere was used.

### Example 7.2

### Subculturing Procedure

Under all circumstances, the cultures were subcultured at 80% confluency or less, and were not allowed to become completely confluent.

The volumes described in this protocol were measured for a 75 cm² flask. For culture vessels of other sizes, amount of dissociation medium used was proportionally reduced or increased.

Culture medium was removed and discarded. The cell layer with 0.25% (w/v) Trypsin-0.53 mM EDTA solution was briefly used to rinse the cells (this removes all traces of serum, which contains trypsin inhibitor). 2.0 to 3.0 ml of Trypsin-EDTA solution was added to the flask and cells were observed under an inverted microscope until cell layer is dispersed. This usually took place within 5 to 10 minutes.

To avoid clumping, the cells were not agitated by hitting or shaking the flask while waiting for the cells to detach. Cells that had difficultly in detaching were placed at 37° C. to facilitate dispersal. About 8.0 ml of complete growth medium and aspirate cells were added by gently pipetting. Appropriate aliquots of the cell suspension were added to new culture vessels. A subcultivation ratio of 1:2 to 1:4 was employed. The cultures were incubated at 37° C.

### Example 7.3

### Medium Renewal

Medium renewal was carried out two times per week.

### Example 7.4

### Complete Growth Medium

The base medium used for this cell line was Dulbecco's Modified Eagle's Medium (DMEM). To make the complete growth medium, fetal bovine serum to a final concentration of 10% was added to the base medium. This medium is formulated for use with 5% CO₂ in air atmosphere. In embodiments where standard DMEM formulations contain 3.7 g/L sodium bicarbonate, 10% CO₂ in air atmosphere is recommended.

### Example 7.5

### Cryoprotectant Medium

Complete growth medium described above were supplemented with 5% (v/v) DMSO.

### EXAMPLE 8

### Cell Culture of HL-1 Cardiomyocytes (Adapted from Claycomb's Lab Protocol)

The culture protocol for HL-1 was kindly provided by Prof. William C. Claycomb (Care of HL-Cardiomyocytes Claycomb Lab). Minor adjustments to the above protocol were done for the current studies.

### Example 8.1

### Making Supplemented Claycomb Medium

| Supplemented Claycomb | | |
|---|---|---|
| Medium | ml | Final Concentration |
| Claycomb Medium | 87 | |
| Fetal bovine serum | 10 | 10% |
| Penicillin/Streptomycin | 1 | 100 U/ml: 100 μg/ml |
| Norepinephrine (10 mM | 1 | 0.1 mM |
| stock) | | |
| L-Glutamine (200 mM stock) | 1 | 2 mM |

The Claycomb medium bottle was wrapped in aluminum foil, since the medium is extremely light sensitive. Supplemented Claycomb medium was kept for two weeks, at which time L-glutamine is replenished.

Norepinephrine [(±)-arterenol] having M.W. 319.3, was prepared using the following. Firstly, norepinephrine was made up in 30 mM ascorbic acid, wherein 100 ml of 30 mM ascorbic acid was prepared by adding 0.59 g ascorbic acid to 100 ml of cell culture grade distilled water. 80 mg norepinephrine was added to 25 ml of the 30 mM ascorbic acid. Norepinephrine was filter-sterilized using a 0.2 μm Acrodisc syringe filter. Aliquots in 1 ml volumes were added into sterile microtubes with screw caps, and stored at −20° C., to form 10 mM (stock) norepinephrine. 1 ml of stock per 100 ml medium was used for a 0.1 mM final concentration. Norepinephrine needed to be made up fresh monthly.

L-Glutamine was provided as a 100× solution, and was aliquoted into working volumes and frozen.

Freezing medium was formed from 95% FBS/5% DMSO. This could be stored up to a week at 4° C.

Soybean Trypsin Inhibitor was prepared using the following. 25 mg of soybean trypsin inhibitor was weighed out, and place into a beaker containing 100 ml of Dulbecco's phosphate buffered saline (PBS; Ca²⁺-free and Mg²⁺-free) until dissolved. The reagent was filter-sterilized using a 0.2 μM syringe filter into a 100 ml bottle. This reagent could be kept for a month at 4° C.

### Example 8.2

### Pre-Coating Flasks with Gelatin/Fibronectin

0.1 g gelatin was weighed out and placed into a 500 ml glass bottle. Distilled water was added to the 500 ml mark, and autoclaved. The gelatin went into solution while being autoclaved to make a final concentration of 0.02%.

Fibronectin was received in a tube as a liquid. 1 ml fibronectin was diluted in 199 ml of 0.02% gelatin. The reagent was mixed gently and immediately aliquoted at 6 ml per 15 ml centrifuge tube. The aliquots were frozen at −20° C.

Before the cells were cultured, tissue culture flasks were coated with gelatin/fibronectin (1 ml/T25 or 3 ml/T75 flask). The flasks were incubated at 37° C. for at least an hour. The gelatin/fibronectin was removed by aspiration just before addition of cells to the flasks.

### Example 8.3

### Culturing Cells

Cultures (5 ml/T25 flask) were fed with supplemented Claycomb Medium every day.

### Example 8.4

### Passaging Procedure for a 1:2 Split

The as-received cells were split when they reach confluency. One of the T25 flasks was split in the ratio 1:2, resulting in two T25 flasks. This set of two T25 flasks constituted the “working” set of cells. The other T25 flask was split in the ratio 1:3, and contents were placed into one T75 flask. After the cells in this T75 flask were confluent, they were split into two T75 flasks. When the cells in these two flasks reached confluency, they were frozen. As mentioned, cultures were split only after full confluence.

Each confluent T25 flask was rinsed briefly with 3 ml of phosphate buffered saline (PBS) warmed to 37° C. (6 ml was used for T75) by pipetting the PBS onto the bottom of the flask (side opposite the cap) while trying not to hit the cells directly. Wash medium was used to rinse gently and was removed by aspiration.

1 ml of 0.05% trypsin/EDTA per T25 flask (3 ml per T75) was added, and incubated at 37° C. for 1 minute. The flasks were removed, and fresh 0.05% trypsin/EDTA was subsequently added. The T25 flasks were incubated for an additional 2 minutes. The contents of the flasks were examined microscopically and, if cells were found to adhere to the flasks, the flasks were rapped on the benchtop to dislodge remaining cells.

An equal amount (1 ml per T25) of soybean trypsin inhibitor was added directly onto cells to inactivate the enzyme. The cells were transferred from the flask into a 15 ml centrifuge tube. The empty flask was rinsed with 5 ml wash medium (Claycomb Medium containing only 5% FBS and penicillin/streptomycin), and added to the cells already in the 15 ml centrifuge tube. The tubes were centrifuged at 500×g for 5 minutes.

Meanwhile, the gelatin/fibronectin solution was removed from each T25 flask, and 4 ml supplemented Claycomb Medium/flask was added. The tube containing the HL-cardiomyocytes was removed from the centrifuge. The supernatant was removed by aspiration, and the pellet gently resuspended in 3 ml of supplemented Claycomb Medium.

1 ml of the cell suspension was transferred into each of three labelled, gelatin/fibronectin-coated T25 flask. Each flask contained 5 ml.

### Example 8.5

### Freezing HL-1 Cells

The contents of one confluent T75 flask were frozen into one cryovial, in order that when cells were needed, this cryovial may be thawed into one T75 flask.

The T75 flask containing the HL-1 culture was briefly rinsed with 5 ml of PBS warmed to 37° C., and removed by aspiration. 3 ml of 0.05% trypsin/EDTA was transferred into the flask. The flask was incubated at 37° C. for 1 minute.

The trypsin/EDTA was removed from the flask, and replaced with 3 ml of fresh 0.05% trypsin/EDTA. The flask was incubated at 37° C. for 2 minutes. A microscope was used to check if the cells were dislodged.

3 ml of soybean trypsin inhibitor was added to the flask, and 6 ml of the solution was transferred into a 15 ml centrifuge tube. Each empty flask was rinsed with 8 ml wash medium, and added to the cells already in the 15 ml centrifuge tube, to amount to a total volume of 14 ml.

The tube was centrifuged for 5 minutes at 500×g. The wash medium was removed by aspiration.

Each pellet was suspended in 1.5 ml of freezing medium (95% FBS/5% DMSO). Pipette was used to resuspend cells into a cryovial. The cryovial, containing the cells was placed into a Nalgene freezing jar containing room temperature isopropanol. The freezing was immediately placed into a −80° C. freezer. The vial was transferred to a liquid nitrogen dewar after twelve hours.

Example 8.6: Thawing HL-1 Cells

A tissue culture flask was gelatin/fibronectin-coated for at least an hour in a 37° C. incubator. The gelatin/fibronectin was removed from the culture flask, and replaced with 10 ml of supplemented Claycomb Medium. Subsequently, the flask was placed back into the incubator. 10 ml of the wash medium was transferred into an empty 15 ml centrifuge tube. The tube was incubated in a 37° C. water bath.

The cells were quickly thawed in a 37° C. water bath for about 2 min, and transferred into the 15 ml centrifuge tube containing the wash medium. The tube was centrifuged for 5 minutes at 500×g. Subsequently, the tube was removed from the centrifuge and the wash medium was removed by aspiration. The pellet was gently resuspended in 5 ml supplemented Claycomb Medium, and added to the 10 ml of medium already in the T75 flask.

The medium was replaced with 15 ml of fresh supplemented Claycomb Medium 4 hours later after attachment of the cells.

### EXAMPLE 9

### F-Actin Staining of Cells on the Optical Discs

To stain for F-actin, cells were fixed in 3.7% paraformaldehyde for 20 mins at room temperature. The cells were then permeabilized for 5 mins with 0.1% Tritin X-100 and blocked with 2% bovine serum albumin for 15 mins at room temperature. Later, the cells were incubated with 200 mg/ml of TRITC-phalloidin (Molecular Probes, USA) for 20 minutes. Microscopy images were acquired with 20× lens on a Zeiss Meta 510 upright confocal microscope. The 3D image stack was reconstructed using LSM Browser.

### EXAMPLE 10

### Measurement of Cell Alignment

Cell outline was determined with the help of an algorithm. The overall cell alignment for an image was found based on the alignment of those outlines (of cells). The direction histogram was then plotted showing the number of cells aligned in particular angles.

### EXAMPLE 11

### Alamar Blue Assay for Cell Proliferation on the Gratings

Alamar blue assay was performed to demonstrate cell proliferation on the gratings. Cells were grown on the Gratings on a dish (CD-R or 2-μm pitch gratings pieces in 24-well microtiter plates). 10 μl of Alamar blue reagent (Life Technologies, USA) was mixed with 90 μl of media and added to each well of the plates, and incubated for 2 hrs. The processed reagent was then transferred to 96-well plates for measurement of fluorescence by Infinite M1000 plate reader (Tecan, Switzerland) using absorption wavelength at 560 nm and emission wavelength at 590 nm. The control used was the Alamar blue reagent mixed with media (kept in wells inside incubator) without any cells.

### EXAMPLE 12

### Micro/Nanogrooves on the Gratings Surface

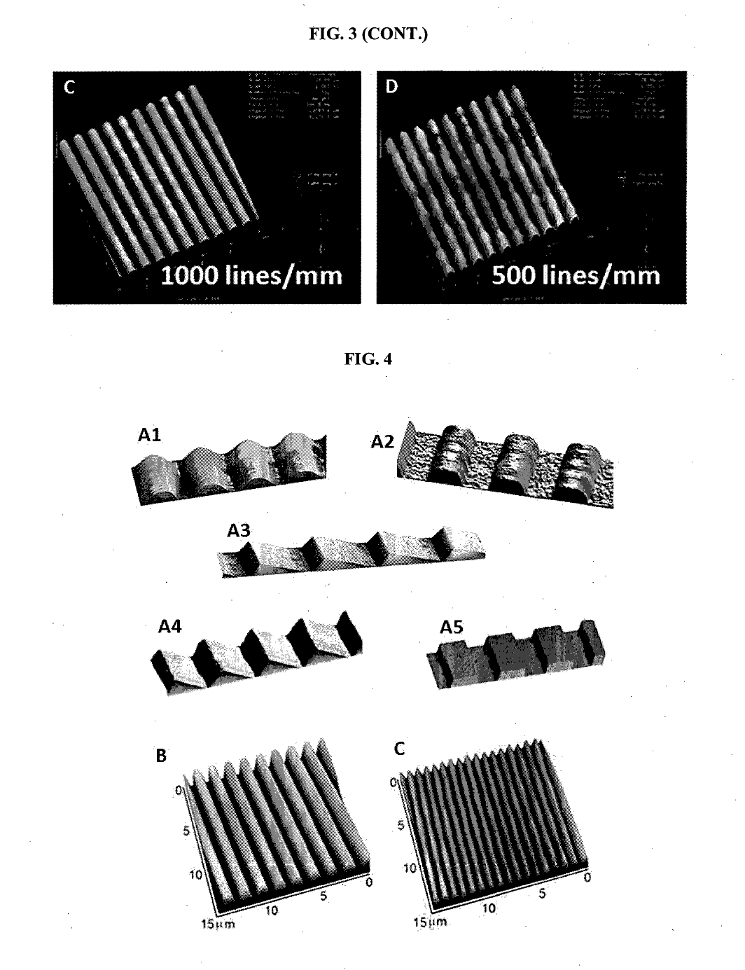

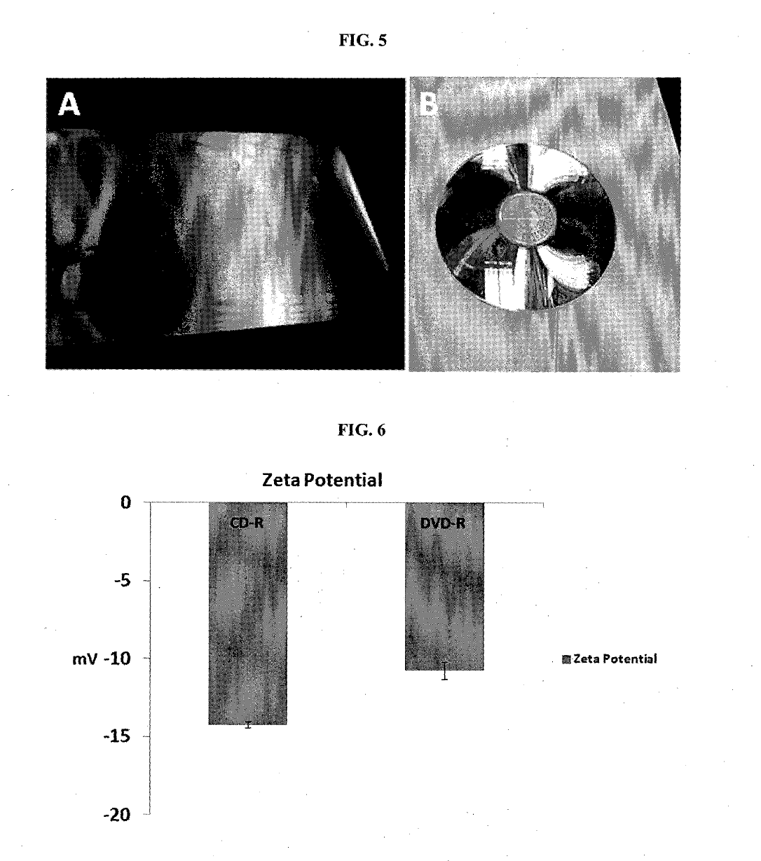

From the SEM images of the CD-R and DVD-R shown in FIGS. 3A and B, parallel grooves may be observed. Two different holographic gratings were observed under AFM (FIGS. 3C and D). For one holographic grating (with 500 lines/mm), the stripe width was found to be 1 μm and stripe height/depth to be 300 nm. For another holographic grating (1000 lines/mm), the stripe width was 500 nm and stripe depth 200 nm. Some of the important parameters of the two holographic gratings and the optical discs (CD-R and DVD-R) and their approximate values are summarized in Table 1.

TABLE 1
Details of the holographic gratings and CD-Rs/DVD-Rs used for the studies

| | | | Stripe width | | | | Stripe | | | | |
|---|---|---|---|---|---|---|---|---|---|---|---|
| | Diffraction | | OR Ridge | | Stripe height | | periodicity | | | | |
| | Gratings | Company | OR Groove width | | OR Depth | | OR Pitch | | Thickness | | Substrate |
| 1 | CD-R | e.g. Imation | 750 | nm | 130 | nm | 1.5 | μm | 1.2 | mm | Polycarbonate |
| 2 | DVD-R | e.g. Imation | 400 | nm | 70 | nm | 800 | nm | 1.2 | mm | Polycarbonate |
| 3 | Holographic | Edmund | 1 | μm | 300 | nm | 2 | μm | ~76 | μm | Polyester |
| | gratings: | optics | | | | | | | | | |
| | 500 lines/mm | | | | | | | | | | |
| | (12700 lines/inch) | | | | | | | | | | |
| 4 | Holographic | Edmund | 500 | nm | 200 | nm | 1 | μm | ~76 | μm | Polyester |
| | gratings | optics | | | | | | | | | |
| | 1000 lines/mm | | | | | | | | | | |
| | (25400 lines/inch) | | | | | | | | | | |

### EXAMPLE 13

### Characterization of the Diffraction Gratings Surface

The zeta potential values of the CD-R/DVD-R surfaces were primarily negative as shown in FIG. 6. Charge interaction between the negatively charged substrate and charged cell may play a role in cell attachment or aggregation.

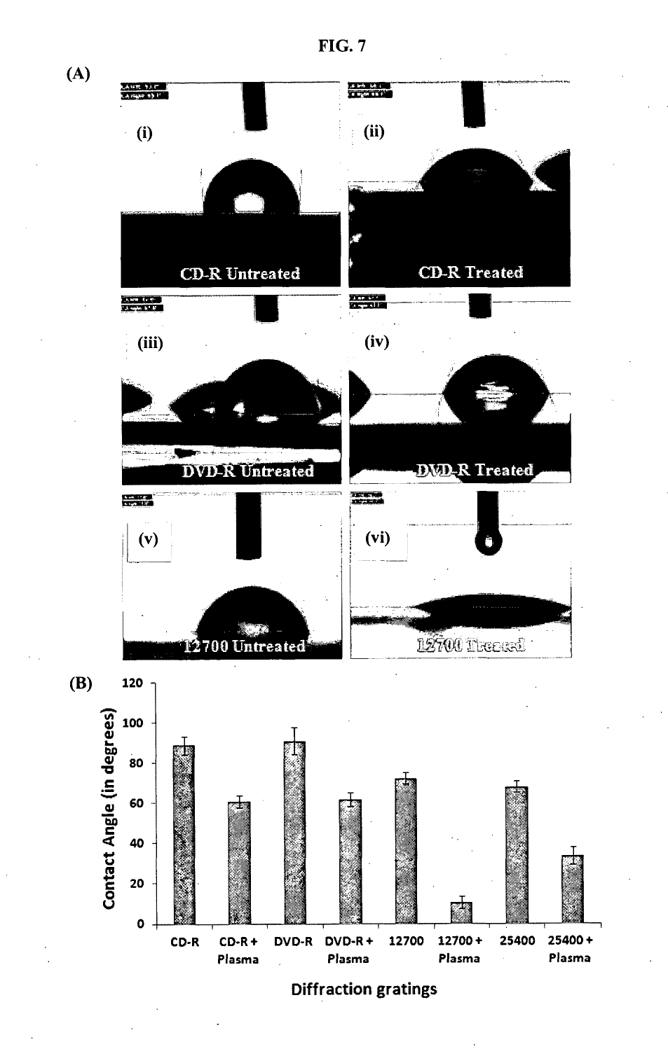

The water contact angles (WCAs) of the CD-R/DVD-R/gratings are shown in FIG. 7. The water contact angles of untreated CD-R and DVD-R obtained were about 90° and for gratings about 70°, i.e. hydrophobic. The oxygen-plasma treated samples showed a decrease in WCAs. Oxygen plasma treatment leads to a higher density of hydroxyl groups on polymeric surfaces. These groups in turn decrease the water contact angle. The decrease in water contact angle shows that there is increase in the hydrophilicity of the surface. As a hydrophilic surface aids in better cell attachment and hence proliferation, by controlling the plasma treatment, hydrophilicity of the surfaces may be modulated.

The surfaces of the gratings may also be modified according to the cell types to be cultured. Ligands like fibronectin, gelatin, and laminin may be coated on the surface prior to the cell culture. This was demonstrated by coating the CD-R surface with a 0.1% gelatin solution. The SEM image in FIG. 8 shows that the surface has gelatin nanospheres and the grooves are not blocked or masked with the spheres.

### EXAMPLE 14

### Cell Attachment and Alignment on the Surface of the Gratings

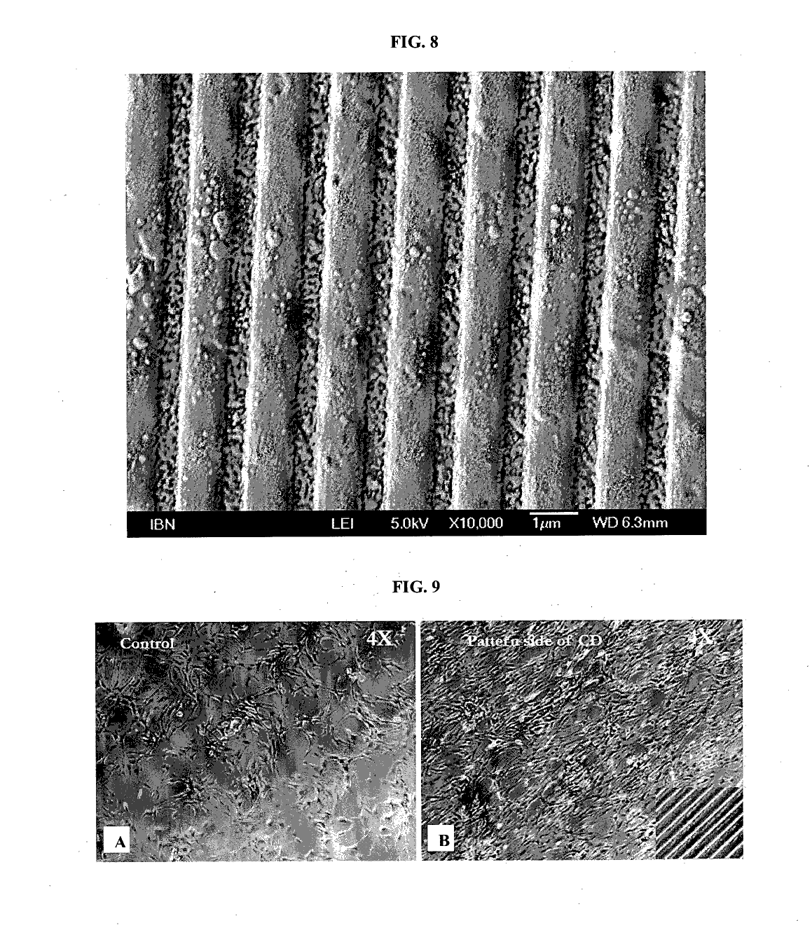

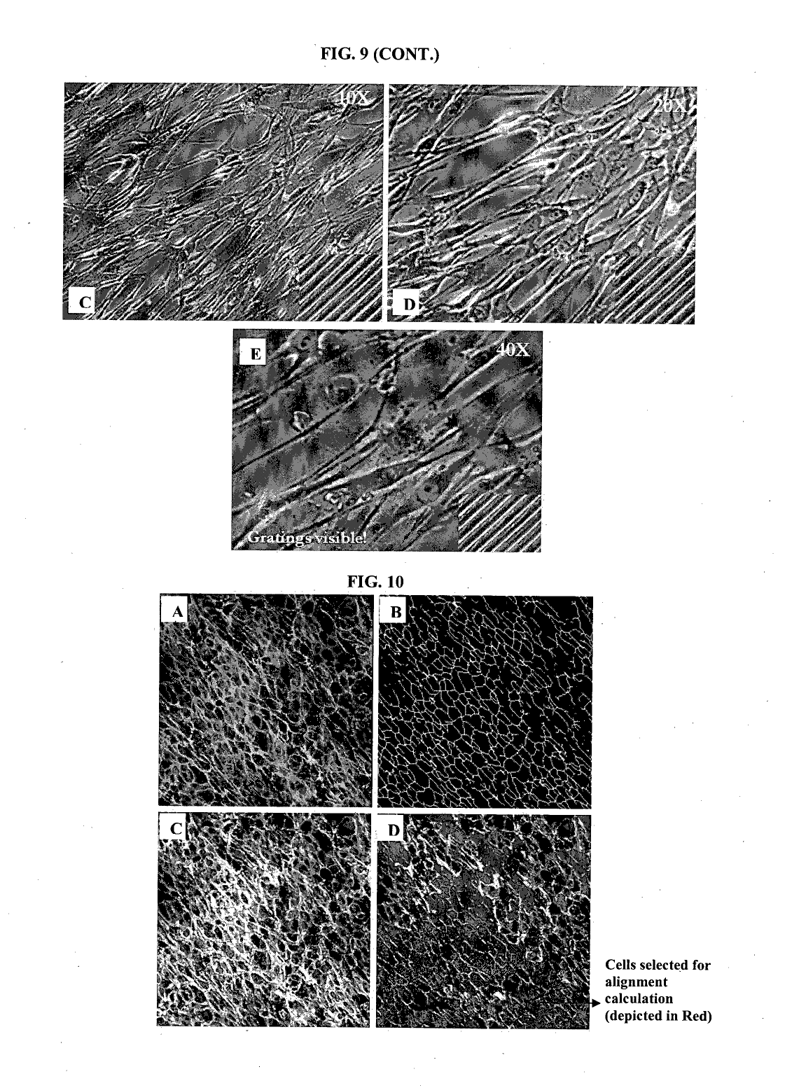

Cells were cultured on gratings surface (gratings on a dish) from 1 day to 5 days for various studies. 3T3 cells, for example, attached to the CD-R surface within 4 hours of seeding and aligned along the grooves in 24 hours (FIG. 9). As seen in the bright-field images, the cells on the polycarbonate control (reverse side of the CD-R with no grooves) spread freely and had no particular alignment, whereas the cells cultured on the grooved surface of the CD-R were aligned along the groove directions (highlighted in the magnified inset figures). The aligned cells demonstrated a stretched morphology (clearer in higher magnification images) and enhanced attachment properties.

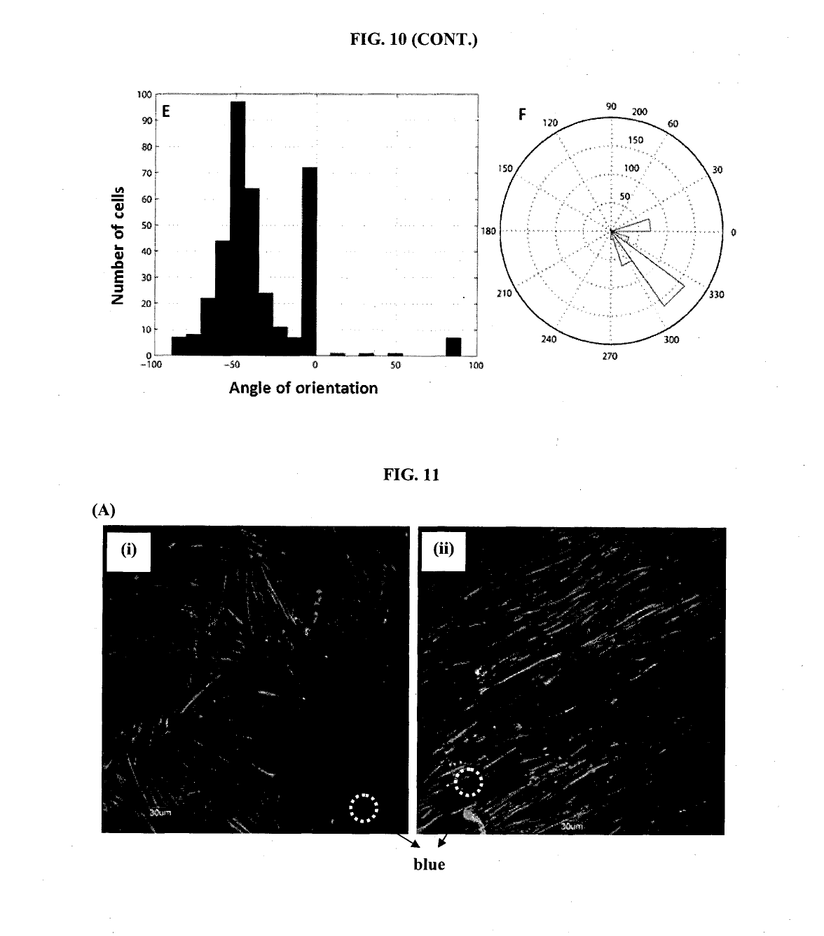

Some preliminary alignment measurements with HL-1 mouse cardiomyocytes grown on CD-R surface were also carried out (gratings on a dish) (FIG. 10A). To find the trend of directionality, the edge of all cells (edge map) was first determined using an algorithm (FIG. 10B). However, for some cases, the edges were disconnected, and hence two or more cells could merge to appear as one cell (with edge). Therefore, to discard those ‘merged cells’, the edge map was superimposed over the actual image of the cells (FIG. 10C). Only those cells for which the edge map co-localized with their actual image were selected (FIG. 10D). As evident from FIGS. 10E and F, the majority of the cells aligned at −50°.

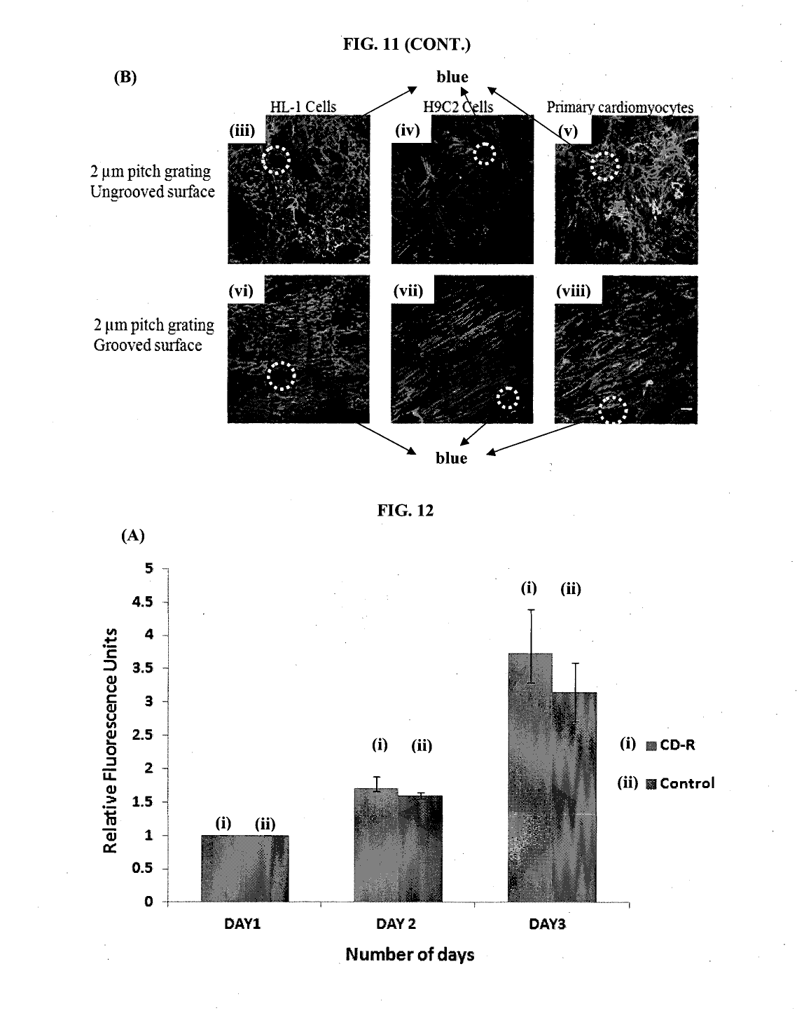

Cells were also cultured on the holographic gratings (24-wells gratings on a dish). FIG. 11 shows the F-actin staining of H9C2 cells being seeded on holographic gratings of 1 μm pitch. The cells seeded on the grooved surface had uniformly aligned actin fibers along the groove direction, whereas on control surface (ungrooved surface of the grating) the cells were randomly oriented.

### EXAMPLE 15

### Cells Showed Similar Proliferation on the Grooves as the Control Surface

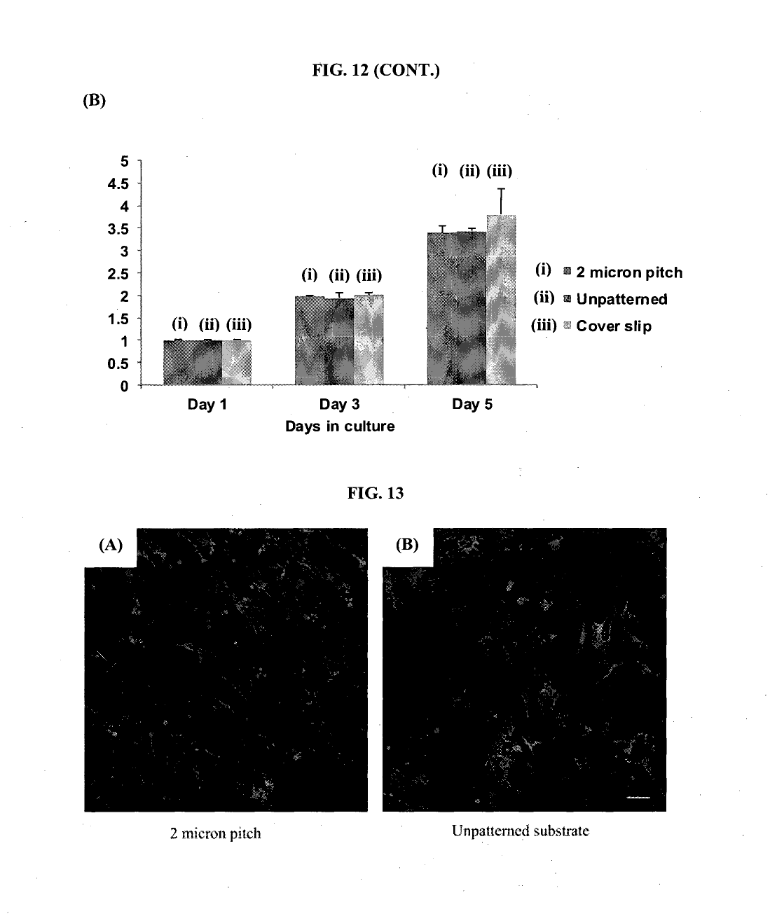

Experiments were carried out to determine whether or not cells grow and respond robustly on the gratings on a dish. FIG. 12 depicts the Alamar blue assay results for 3T3 cells being cultured on CD-R surface. The assay was performed on 1st, 2nd and 3rd day after the cell seeding on the CD-R. The fluorescence readings were normalized to the first day for each configuration of CD-R sample and the ungrooved CD-R control. The cell proliferation on the grooved surface and the unpatterned control appeared to be similar or better with no significant difference on 2nd or 3rd day. This implies the gratings surface does not hinder cell growth in any way.

### EXAMPLE 16

### Differentiation of C2C12 on Diffraction Gratings

The skeletal myoblast cell line C2C12 cells were seeded at a density of 50,000 cells per well in the 24-well gratings on dish (for all the three CD-R, DVD-R and optical gratings) plates, and grown in the proliferation medium which consists of DMEM low glucose with 10% FBS. After 2 days, at 90% to 100% confluency, the medium was switched to differentiation medium which contains 1% FBS and 1% insulin-transferrin-sodium selenite, and was changed daily for another 3 consecutive days. At the end of the 3 days, the cells were fixed for myosin heavy chain (MHC) and F-actin. Differentiation was assessed by taking 10 random images from each sample and quantifying the percentage of cells that picked up the MHC marker. This was done by comparing the ratio of the red channel (MHC) which only stained the cells that were differentiated and maturing to the green channel (F-actin) which stained all the cells for each image cells.

Differentiation of C2C12, a rat derived myoblast cell line, was induced by reducing the fetal bovine serum to 1% in the presence of insulin-selenium. After 3 days, the cells were fixed and stained for F-actin and myosin heavy chain. The differentiation was assessed by the amount of cells that were stained with the MHC antibody. The experiment was repeated three times and 10 random images were taken for each platform.

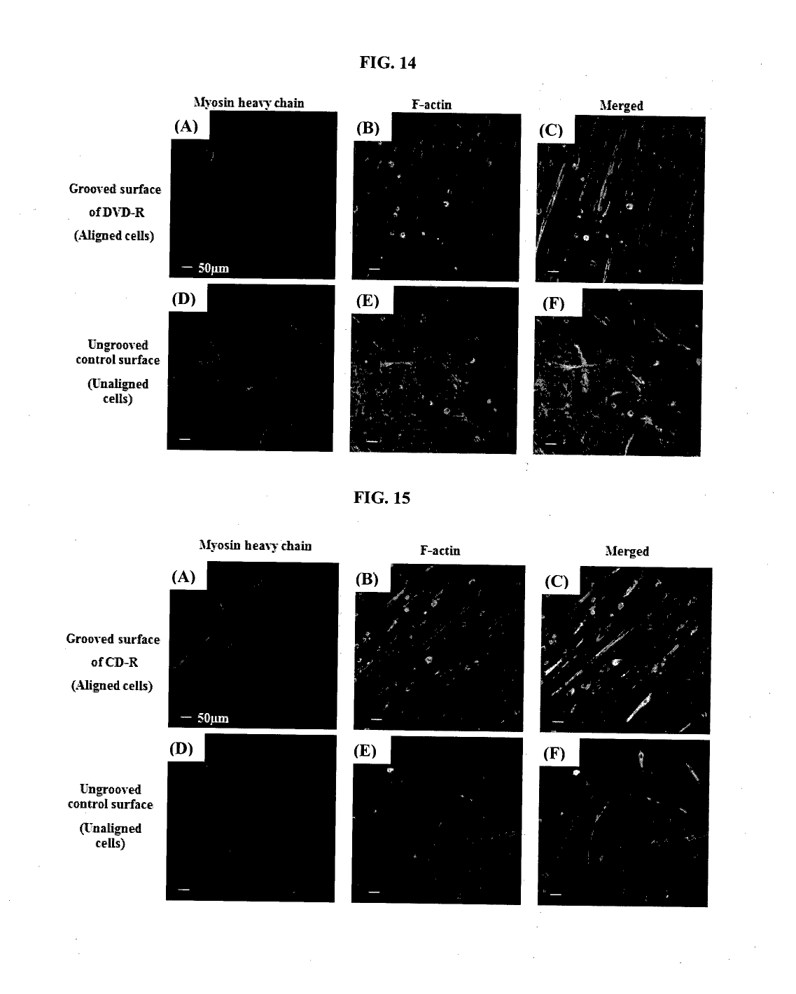

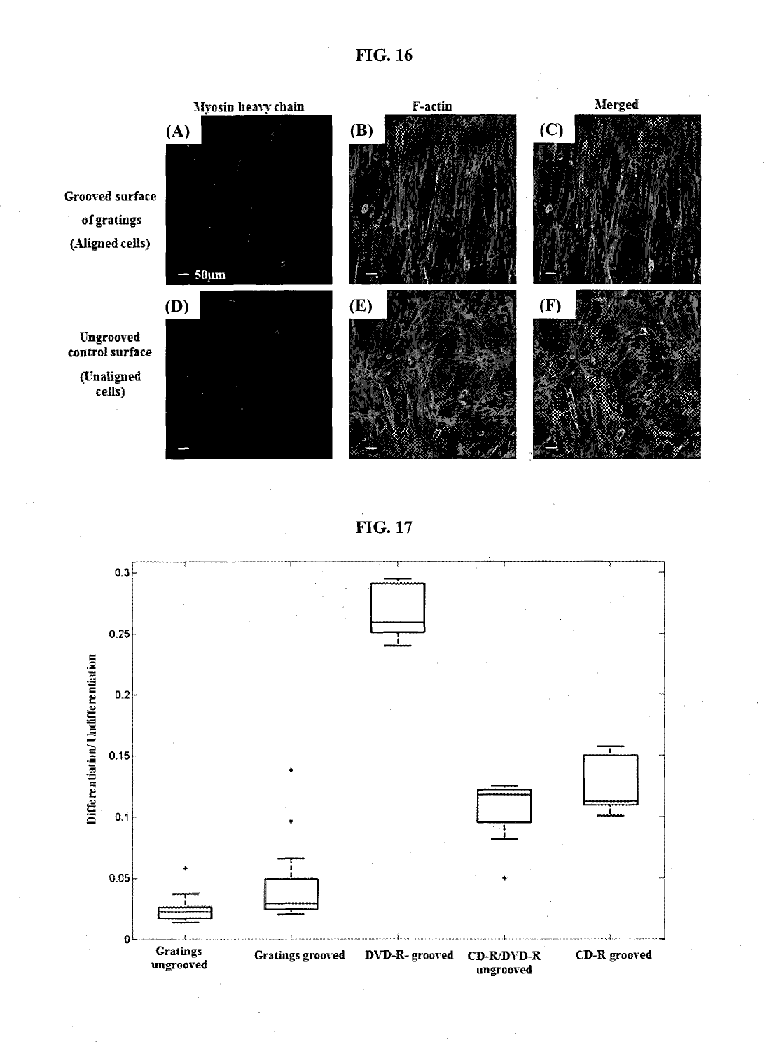

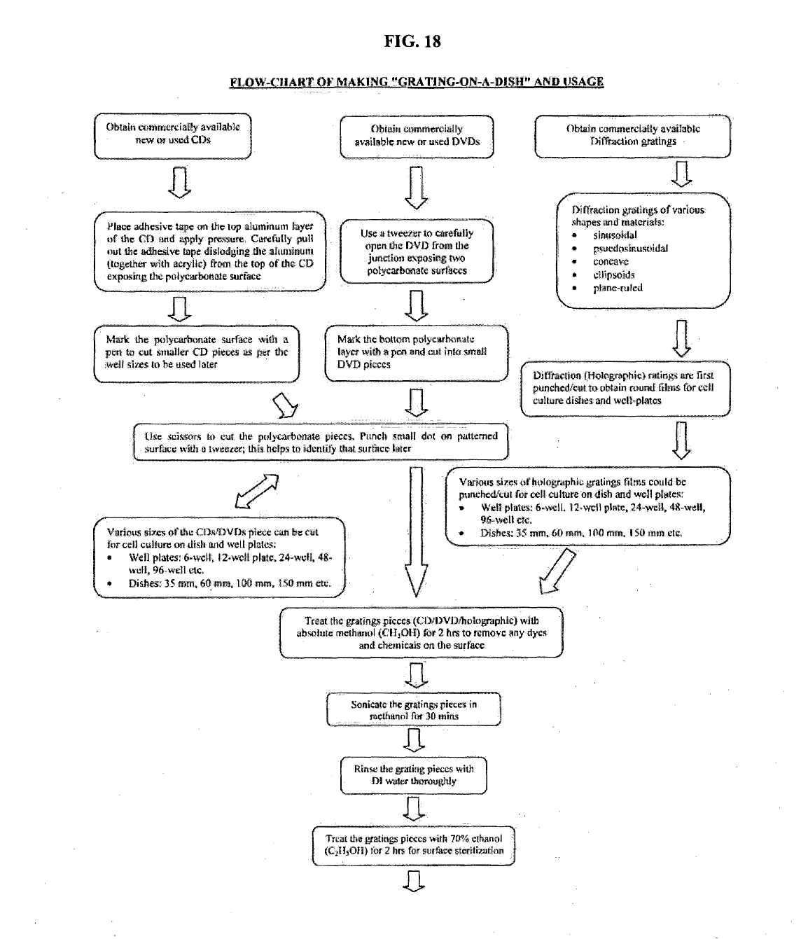

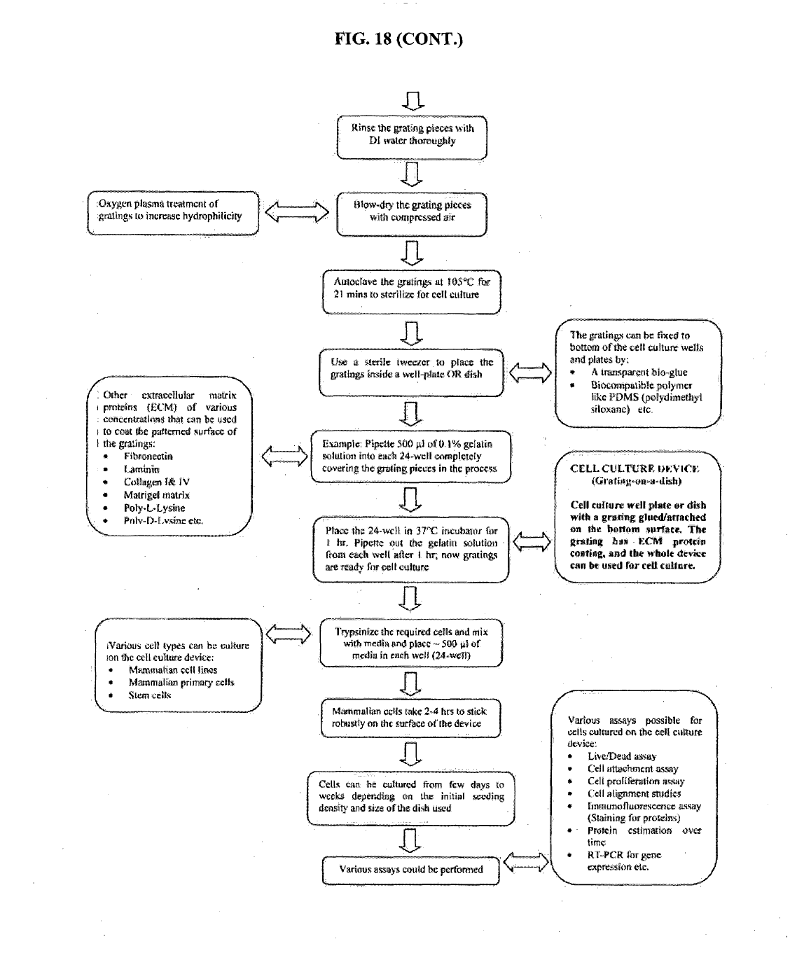

The result obtained demonstrates that cell alignment enhances differentiation and maturation of C2C12 cells. It was found that more cells cultured on the side with the grooves/ridges were expressing the myosin heavy chain when compared to the side without the grooves/ridges. Interestingly, it was noted that overall, the cells cultured on the DVD-R were not only expressing the MHC but had also fused to form more myotubes. This indicates that there was enhanced differentiation and maturation of the cells on the DVD-R and suggests that cells are sensitive to both groove dimensions and material properties. This difference between the CD-R/DVD-R and the holographic gratings may be due to material properties while the difference between the CD-R and DVD-R may be due to the groove dimensions. C2C12 cells have been shown to be sensitive to surface charges and material stiffness. The data shown in FIGS. 14 to 16 shows the immunostaining of the C2C12 cells after 3 days in differentiation media. FIG. 17 shows a computational analysis of the average differentiation rate across all platforms.

“Gratings on a dish” devices for culturing and aligning cells thereon have been developed. Optical media such as holographic gratings and optical discs comprising diffraction gratings may be cut to appropriate sizes and placed in cell culture dishes and well plates. Physical characterization of the gratings revealed parallel grooves on the optical media. The optical media comprising the gratings may be treated with organic solvents and rinsed with DI water to render the surface suitable for cell culture. In various embodiments, the gratings surface was made more hydrophilic with oxygen plasma for better cell attachment and proliferation. The gratings may incorporate ECM components to make the surface more physiologically relevant for various cell types.

Various cell types from rat and mouse species have been cultured on the gratings on a dish, and shown to align on the patterned surface. For example, cardiomyocytes were shown to survive, and to attach and proliferate on the patterned surface. Quantification of the alignment also indicated the preferential alignment of cells along the groove direction. The growth of the cells on the grooved surface occurred with the same robustness as the ungrooved surface. The skeletal myoblast cell line C2C12 cells showed enhanced differentiation and maturation on the grooved surface of the DVD-R compared to the CD-R or holographic gratings, suggesting that cells are sensitive to both groove dimensions and material properties.

Gratings on a dish may potentially be used for drug testing applications. Cells that need alignment for maturation, like the cardiomyocytes, may be cultured or grown on this device with an enhanced physiological state (matured phenotype) for pharmacological investigation. Freshly isolated cardiomyocytes or neurons from animal models may be aligned on the gratings and remodeled back to the matured phenotype. For primary cardiomyocytes, electrical-field stimulation may be used in tandem with the forced alignment due to the gratings. These phenotypes would be most suitable for practical drug testing applications.

Methods to culture cell or tissue according to various embodiments of the invention include use of commercially available optical media for direct cell culture, which is faster and more cost effective compared to current state of the art methods, and may be adapted by laboratories and start-ups for drug testing and other biomedical applications. Apart from drug testing applications, other direct uses of the device include applications in tissue engineering, stem-cell differentiation and mechanobiological studies.

1-40. (canceled)

41. A method of culturing cells or tissues, the method comprising:
a) providing a support, wherein the support is an optical medium with a patterned surface, wherein the patterned surface of the optical medium comprises or consists essentially of pre-grooves;
b) contacting the support with cells and a culture medium; and
c) culturing the cells under suitable cultivation conditions.

42. The method according to claim 41, wherein the cultured cells or tissues are at least substantially aligned with the patterned surface of the optical medium.

43. The method according to claim 41, wherein the patterned surface of the optical medium comprises a grating.

44. The method according to claim 43, wherein the grating is a diffraction grating.

45. The method of claim 41, further comprising treating the patterned surface prior to operation b) to render the surface suitable for cell growth.

46. The method according to claim 45, wherein treating the patterned surface comprises contacting the patterned surface with a solvent to remove organic material from the patterned surface.

47. The method according to claim 45, wherein treating the patterned surface comprises contacting the patterned surface with a disinfectant or autoclaving to sterilize the patterned surface.

48. The method according to claim 45, wherein treating the patterned surface comprises treating the patterned surface with an ionized oxygen-containing plasma.

49. The method according to claim 45, wherein treating comprises exposing a patterned surface of an optical medium.

50. The method according to claim 49, wherein exposing comprises physically removing layers of material coated on the patterned surface.

51. The method according to claim 41, wherein the optical medium is selected from the group consisting of CDs, CD-Rs, CD-RWs, DVDs, DVD-Rs, DVD-RWs, DVD+Rs, DVD+RWs, BDs, BD-Rs, BD-REs, laser discs, mini discs, hybrid discs, and holographic gratings.

52. The method according to claim 41, wherein the optical medium is selected from the group consisting of CD-Rs, CD-RWs, DVD-Rs, DVD-RWs, DVD+Rs, DVD+RWs, BD-Rs, BD-REs, laser discs, mini discs, hybrid discs, and holographic gratings.

53. The method according to claim 41, wherein the patterned surface of the optical medium comprises grooves which are arranged at least substantially parallel to one another.

54. The method according to claim 41, wherein the optical medium comprises or consists of a biocompatible material.

55. The method according to claim 41, wherein the optical medium comprises or consists of polymer, glass, silicon, metal, alloys, metal oxides, metal carbides, metal fluorides, or mixtures thereof.

56. The method according to claim 55, wherein the polymer is selected from the group consisting of polycarbonate, polyester, polystyrene, poly(methyl methacrylate), polyurethane, polyacrylic acid, derivatives thereof and copolymers thereof.

57. The method according to claim 41, wherein the cells comprise or consist essentially of mammalian cells.

58. The method according to claim 41, wherein the cells are selected from the group consisting of organ cells, muscle cells, nerve cells, stem cells, epithelial cells, connective tissue cells, cancerous cells (cell lines), and combinations thereof.

59. The method according to claim 41, wherein the cells are selected from the group consisting of human embryonic stem cells (hECs), human mesenchymal stem cells (hMSCs), keratinocytes, tenocytes, ligament cells, cardiomyocytes, osteoblasts, fibroblasts, myoblasts, endothelial cells, and combinations thereof.

60. The method according to claim 41, wherein the cells are selected from the group consisting of Chinese Hamster Ovary (CHO) cells, COS cells, HL-1 cells, H9C2 cells, 3T3 cells, C2C12 cells, PC12 cells, NIH3T3 cells, HeLa cells, and combinations thereof.

61. The method according to claim 41, wherein the optical medium is a recycled optical medium.

62. A cell or tissue culture device comprising an optical medium, wherein a patterned surface of the optical medium forms a support upon which cells are cultivated, wherein the patterned surface of the optical medium comprises or consists essentially of pre-grooves.

63. The device according to claim 62, wherein cells or tissues cultivated using the device are at least substantially aligned with the patterned surface of the optical medium.

64. The device according to claim 62, wherein the patterned surface of the optical medium comprises a grating.

65. The device according to claim 64, wherein the grating is a diffraction grating.

66. The device according to claim 65, wherein the diffraction grating comprises alternating protrusions and grooves, wherein the alternating protrusions and grooves are arranged at least substantially parallel to one another.

67. The device according to claim 66, wherein the protrusion has a height of between about 70 nm to about 300 nm.

68. The device according to claim 66, wherein the protrusion or groove has a width of between about 400 nm to about 1 μm.

69. The device according to claim 65, wherein the diffraction grating has a period of between about 800 nm to about 2 μm.

70. The device according to claim 62, wherein the optical medium is selected from the group consisting of CDs, CD-Rs, CD-RWs, DVDs, DVD-Rs, DVD-RWs, DVD+Rs, DVD+RWs, BDs, BD-Rs, BD-REs, laser discs, mini discs, hybrid discs, and holographic gratings.

71. The device according to claim 62, wherein the optical medium is selected from the group consisting of CD-Rs, CD-RWs, DVD-Rs, DVD-RWs, DVD+Rs, DVD+RWs, BD-Rs, BD-REs, laser discs, mini discs, hybrid discs, and holographic gratings.

72. The device according to claim 62, wherein the patterned surface of the optical medium comprises grooves which are arranged at least substantially parallel to one another.

73. The device according to claim 62, wherein the optical medium comprises or consists of a biocompatible material.

74. The device according to claim 62, wherein the optical medium comprises or consists of polymer, glass, silicon, metal, alloys, metal oxides, metal carbides, metal fluorides, or mixtures thereof.

75. The device according to claim 74, wherein the polymer is selected from the group consisting of polycarbonate, polyester, polystyrene, poly(methyl methacrylate), polyurethane, polyacrylic acid, copolymers thereof, and combinations thereof.

76. The device according to claim 62, wherein the cells are selected from the group consisting of organ cells, muscle cells, nerve cells, stem cells, epithelial cells, connective tissue cells, cancerous cells (cell lines), and combinations thereof.

77. The device according to claim 62, wherein the cells are selected from the group consisting of human embryonic stem cells (hECs), human mesenchymal stem cells (hMSCs), keratinocytes, tenocytes, ligament cells, cardiomyocytes, osteoblasts, fibroblasts, myoblasts, endothelial cells, and combinations thereof.

78. The device according to claim 62, wherein the cells are selected from the group consisting of Chinese Hamster Ovary (CHO) cells, COS cells, HL-1 cells, H9C2 cells, 3T3 cells, C2C12 cells, PC12 cells, NIH3T3 cells, HeLa cells, and combinations thereof.

79. The device according to claim 62, wherein the optical medium is a recycled optical medium.

80. The device according to claim 62, wherein the device is a cell culture dish or a well plate or a flask.
